(19) United States
(12) Patent Application Publication
(10) Pub. No.: US 2018/0256762 A1
(43) Pub. Date: Sep. 13, 2018

(54) STERILIZATION AND CLEANING DEVICE FOR MULTIPLE DENTURES

(71) Applicant: HA YE CO., LTD, Daejeon (KR)

(72) Inventor: Young Su KIM, Daejeon (KR)

(73) Assignee: HA YE CO., LTD, Daejeon (KR)

(21) Appl. No.: 15/756,306

(22) PCT Filed: Sep. 29, 2016
(86) PCT No.: PCT/KR2016/010901

§ 371 Date: Feb. 28, 2018

(30) Foreign Application Priority Data
Oct. 1, 2015 (KR) 10-2015-0138303
Jun. 22, 2016 (KR) 10-2016-0077751

(51) Int. Cl. A61L 2/07; A61L 2/18; A61L 2/025; B08B 3/12

(52) U.S. Cl. CPC A61L 2/07; A61L 2/18; A61C 17/036; B08B 3/12; A61L 2/025

(57) ABSTRACT

The present invention relates to a cleaning/sterilization and storage device for a plurality of dentures, which is capable of simultaneously cleaning a plurality of dentures and, more particularly, to a cleaning/sterilization and storage device for a plurality of dentures, which is capable of simultaneously cleaning a large number of dentures collected from facilities where there are many denture users such as a nursing home or a geriatric hospital to supply the same to the user again, and killing germs as well as separating foreign substances to supply the dentures to the user.

### TECHNICAL FIELD

The present invention relates to a denture sterilizing and cleaning device, and more particularly, to a sterilizing and cleaning device for a plurality of dentures capable of simultaneously cleaning a plurality of dentures.

More particularly, the present invention relates to a sterilizing and cleaning device for a plurality of dentures, which is capable of simultaneously cleaning a large number of dentures collected from facilities where there are many denture users such as a nursing home or a geriatric hospital to supply the dentures to the users again, and sterilizing germs as well as separating foreign substances to supply the dentures to the users.

### RELATED ART

With the advancement of medical technology, a variety of medical apparatuses have been developed, and out of dental medical technologies, artificial teeth have been developed and used.

Dentures are representative artificial teeth.

Dentures are a prosthetic device for replacing missing teeth and artificial teeth consisting of metals and plastic. Like teeth, such a denture needs to be often cleaned and managed to be kept clean. A common method of managing a denture is removing a denture after a meal or before bed, brushing it and storing it in a cleaning solution or clean water.

However, such a method is problematic because a container for storing a denture is opened thereby increasing the risk of inflow of foreign substances or spill of a cleaning solution or pure water. In particular, an old denture needs to be often sterilized for hygiene since it creates an environment where germs invisible to the naked eyes breed and causes a bad smell. Even though hot water disinfection is the most effective way to sterilize a denture, this might cause deformation of the denture because of the heat. In general, a disinfectant is mixed with water and then, bubbles are made to soak a denture for a certain period of time. However, this causes inconvenience to the users and disinfectants are not easy to obtain. It is difficult for the infirm to sterilize their dentures in this way. In particular, it is hard for the infirm in a nursing home or a geriatric hospital to sterilize and clean their dentures by themselves. They need help from others to do this, but, even so, dentures are not sterilized and cleaned well.

Improved technologies for sterilizing and managing dentures more easily have been developed. For instance, there are Patent Document 1 (Korean Patent No. 0870775), Patent Document 2 (Korean Patent No. 0770628) and Patent Document 3 (Korean Patent No. 0822833).

Patent Document 1 relates to a separable ultrasonic cleaner in which a main body and a cleaning tub are separable, and the cleaning tub, at the center of the lower plate thereof, has a center terminal and a ring terminal, and the separable ultrasonic cleaner includes a plate spring in which the outer side of the ring terminal at the lower plate of the cleaning tub is formed with an arc-type cut line of the inner side of a concentric circle and with an arc-type cut line of the outer side of a concentric circle, and one side of the cut line of the inner side and one side of the cut line of the outer side are connected by a cut line again such that the center portion of the lower plate may elastically move upwards and downwards.

Patent Document 2 relates to a denture cleaning and sterilizing device having a low temperature plasma generator includes a device in which a body engages the upper surface of a main body, the body, on the upper surface thereof, has a lid installed to open and close, and the main body, at one side of the upper portion thereof has a toothbrush inserting hole, a device installing a low temperature plasma generator in which the main body has an engaging hole therein, and a multistage-type inserting hole is formed in the engaging hole and at the center portion of the inside of the body, electrodes are placed in this inserting hole, an intercepting plate is inserted between electrodes, each electrode has a ground bolt engaging a packing to protrude from the lower end of the main body, a fixer for fixing electrodes and a filter are placed at the upper portion of the electrodes, at the upper portion of the filter is a lid, on the upper surface thereof, having a couple of through-holes formed at regular intervals and fixedly installed at the main body by means of a screw, the ground bolt protruding from the lower surface of the main body engages a packing for preventing water leakage and then engages a ground nut to be fixed, and a device in which a denture containing container is built in a main body.

Patent Document 3 includes a container for containing cleaning water and a denture; a main body provided at one side of the container and supporting the container; a fluid rotating device for generating rotating power to rotate the cleaning water in the container; a cleaning pin provided in the container and rotated by the fluid rotating device to remove foreign substances between the teeth of a denture; a sterilizing device provided in the container and sterilizing the denture by using electrolysis; and a controller provided at one side of the main body and controlling the operation of the sterilizing device and the fluid rotating device, and the container is separable from the main body.

These conventional denture cleaning and storing devices simultaneously clean and sterilize a denture and make it easy to store the denture.

### DETAILED DESCRIPTION OF THE INVENTION

### Technical Problem

Conventional denture cleaning devices are used for cleaning an individual denture, and this makes it hard for facilities such as a nursing home or a geriatric hospital, where there are many denture users who have difficulty moving freely, to store and clean a large number of dentures.

In particular, because the users living in a nursing home or a geriatric hospital have declining health and intelligence, they cannot use a denture cleaning and storing device. Instead of the users, managers such as carers or caregivers etc. need to clean and store the dentures. However, cleaning and sterilizing dentures is unpleasant to them, and cleaning a large number of dentures manually causes many minute grooves such as scratches on the surfaces of the dentures thereby creating an environment where germs breed and leading to secondary contamination. Due to this, it is difficult to manage a large number of dentures.

As a means to solve the problems with the related arts, the present invention is directed to providing a sterilizing and cleaning for a plurality of dentures capable of simultaneously sterilizing and cleaning a plurality of dentures.

More particularly, the present invention is directed to providing a sterilizing and cleaning device for a plurality of dentures, which is capable of simultaneously sterilizing and cleaning a large number of dentures collected from facilities where there are many denture users such as a nursing home or a geriatric hospital to supply the dentures to the users again, and sterilizing germs as well as separating foreign substances to supply the dentures to the users.

### Technical Solution

As a means to achieve the aim described above, a sterilizing and cleaning device for a plurality of dentures according to the present invention, which is capable of simultaneously sterilizing and cleaning a plurality of dentures, has a sterilizing and cleaning tub which is provided with a plurality of compartments into which denture boxes storing dentures are detachably put, and stores sterilizing and cleaning water; and a bubble sterilizing cleaner including at least one bubble generator for cleaning the dentures stored in the denture boxes submerged in the sterilizing and cleaning water by supplying air bubbles to each compartment of the sterilizing and cleaning tub.

Another aspect of a sterilizing and cleaning device for a plurality of dentures capable of simultaneously cleaning a plurality of dentures includes a sterilizing and cleaning tub for storing sterilizing and cleaning water therein; at least one bubble generator for cleaning dentures stored in denture boxes submerged in the sterilizing and cleaning water by supplying air bubbles to each compartment of a box supporting means, described hereunder, installed at the sterilizing and cleaning tub; and a box supporting means having a plurality of compartments which are accessibly installed at the sterilizing and cleaning tub and at which the denture boxes storing the dentures are detachably installed.

Preferably, the sterilizing and cleaning tub, at the upper portion thereof, further includes a light sterilizer for sterilizing germs left on the dentures by irradiating light on the dentures.

Further, a bubble sterilizing cleaner, at one side thereof, may further include a display for showing advertisement descriptions or informing how to use the cleaning device.

The bubble sterilizing cleaner, at one side thereof, further includes a high pressure steam cleaner which separates foreign substances left on the dentures by spraying high pressure steam on the dentures, and the high pressure steam cleaner includes a steam spraying means, at the inner wall thereof, provided with a plurality of spraying nozzles to spray high pressure steam supplied through a supplying hose; and a supporter installed at the lower portion of the steam spraying means for supporting the dentures.

Further, a sterilizing and cleaning for a plurality of dentures capable of simultaneously cleaning a plurality of dentures further includes a sterilizing/storing device, and the sterilizing/storing device may include a sterilizing tub which stores an antiseptic solution therein; one or more of antiseptic solution generators installed at one side of the sterilizing tub; a box supporting means which is accessibly installed at the sterilizing tub and at which the denture boxes storing the dentures are detachably installed; a light sterilizer installed at the upper portion of the box supporting means and sterilizes germs left on the dentures by irradiating light on the dentures.

The high pressure steam cleaner is movably installed at the upper portion of the sterilizing/storing device, the bubble sterilizing cleaner is installed at the upper portion of the high pressure steam cleaner and uses the sterilizing tub, included in the sterilizing/storing device, as a sterilizing and cleaning tub by storing sterilizing and cleaning water therein, the box supporting means, at the upper portion thereof, has steam spraying nozzles included in the high pressure steam cleaner, the steam spraying nozzles are installed between the bubble sterilizing cleaner and the antiseptic solution generator, and the box supporting means is installed to be ascended by using an ascending means such that the dentures move among the sterilizing and cleaning tub, the light sterilizer and the steam spraying nozzles to be cleaned and sterilized.

The box supporting means is installed to be ascended by an ascending means. The box supporting means ascends such that germs on the dentures are sterilized by light irradiated by the light sterilizer when light is used for sterilization, and the box supporting means descends to be submerged in the antiseptic solution stored in the sterilizing tub when the dentures are stored. Then, preferably, the dentures are stored in the state where the dentures are sterilized by the antiseptic solution converted by the antiseptic solution generator.

Further, the sterilizing tub is divided into a plurality of compartments, and the box supporting means may be configured to have a net shape formed with denture box installing holes to put a denture box into each compartment.

### Advantageous Effects

As described above, a sterilizing and cleaning device for a plurality of dentures according to the present invention is capable of simultaneously cleaning and sterilizing a large number of dentures such that facilities where there are many denture users such as a nursing home or a geriatric hospital may simultaneously clean and sterilize a large number of dentures and store the dentures in an aseptic state.

### DESCRIPTION OF THE DRAWINGS

FIG. 1 is a perspective view of an embodiment of a sterilizing and cleaning device for a plurality of dentures according to the present invention.

FIG. 2 is a front sectional view of an embodiment of a sterilizing and cleaning device illustrated in FIG. 1.

FIG. 3 is a front sectional view of another embodiment of a sterilizing and cleaning device illustrated in FIG. 1.

FIG. 4 is a front sectional view of another embodiment of a sterilizing and cleaning device for a plurality of dentures according to the present invention.

FIG. 5 is a perspective view of an embodiment of a high pressure steam cleaner included in a sterilizing and cleaning device for a plurality of dentures according to the present invention.

FIG. 6 is a sectional view of embodiments different from each other of a high pressure steam cleaner included in a sterilizing and cleaning device for a plurality of dentures according to the present invention.

FIG. 7 is a perspective view of an embodiment of a bubble sterilizing cleaner included in a sterilizing and cleaning device for a plurality of dentures according to the present invention.

FIG. 8 is an exploded perspective view of an embodiment of a denture box provided to a sterilizing and cleaning device for a plurality of dentures according to the present invention.

FIG. 9 is a perspective view of an embodiment of a box supporting means provided to a sterilizing and cleaning device for a plurality of dentures according to the present invention.

FIG. 10 is a bottom view of an embodiment of a steam spraying means included in a high pressure steam cleaner installed in a sterilizing and cleaning device for a plurality of dentures illustrated in FIG. 9.

FIG. 11 is an exploded perspective view of an embodiment of a cleaning brush provided to a sterilizing and cleaning device for a plurality of dentures according to the present invention.

FIG. 12 is a front view of an embodiment of a cleaning brush provided to a sterilizing and cleaning device for a plurality of dentures according to the present invention.

### MODE FOR CARRYING OUT THE INVENTION

The present invention may be modified and be embodied in various different ways. Specific embodiments will be illustrated in the drawings and will be described in detail in the detailed description of the invention. However, it is to be understood that the embodiments are not meant to limit the invention to the described embodiments, but, on the contrary, are intended to cover various modifications, equivalents, and alternatives included within the spirit and scope of the present invention.

While each drawing is described, similar reference numerals are used to refer to similar elements. When detailed description of the well-known technology to which the invention pertains makes the gist of the invention vague, the detailed description will be excluded.

Hereunder, preferred embodiments of the present invention will be described in detail by reference to the drawings.

A sterilizing and cleaning device for a plurality of dentures according to the present invention is characterized by simultaneously cleaning and sterilizing a plurality of dentures 100.

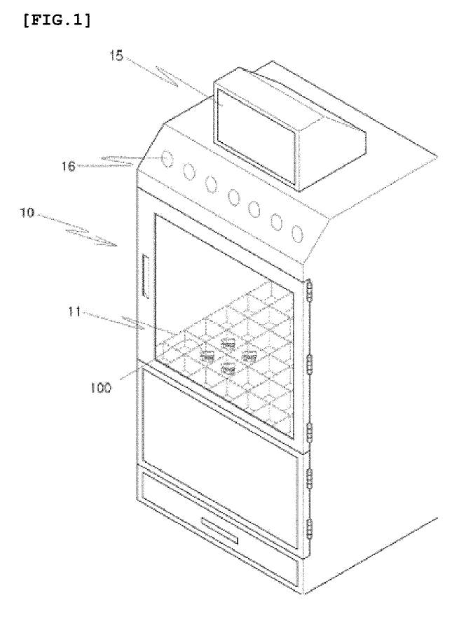

Each denture needs to be provided with an identifiable sign such as a name tag because dentures 100 are cleaned and sterilized at once when a sterilizing and cleaning device for dentures according to the present invention is used to clean and sterilize the dentures, and the identifiable sign may be engraved at one side of the dentures except the artificial teeth portion thereof. A sterilizing and cleaning device for dentures according to the present invention basically has a bubble sterilizing cleaner 10 illustrated in FIGS. 1 to 3.

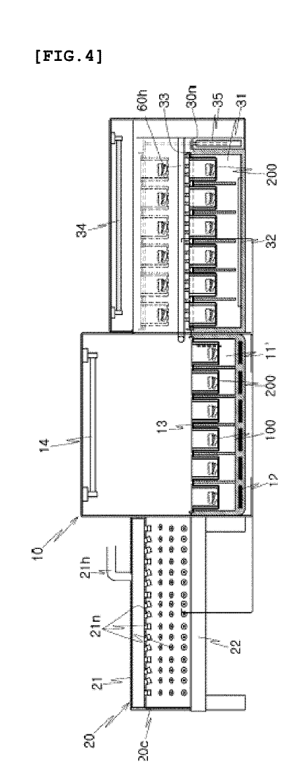

The bubble sterilizing cleaner 10, as illustrated in FIGS. 1 to 3, may be a separate unit, or as illustrated FIG. 4, may be formed integrally with other devices.

The bubble sterilizing cleaner 10 provides bubbles to sterilizing and cleaning water to separate foreign substances from the dentures by using force exerted by ascending or bursting bubbles to cause turbulence in the sterilizing and cleaning water, or by causing force exerted by bursting bubbles to act on the denture 100.

To simultaneously clean a plurality of dentures, the bubble sterilizing cleaner 10 has a plurality of compartments such that the plurality of dentures may be stored at once, but may not be mixed up.

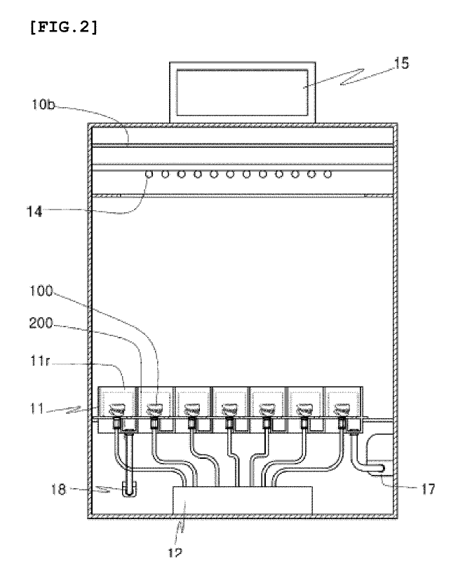

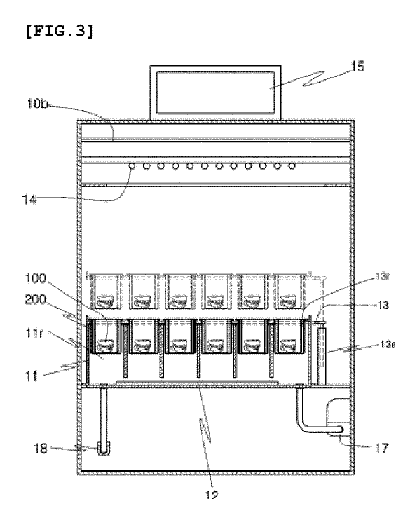

That is, the bubble sterilizing cleaner 10 may be configured as illustrated in FIG. 2 or FIG. 3 depending on the way that the compartments are formed.

As in FIG. 2 illustrating an embodiment of compartments 11r integrally installed in a sterilizing and cleaning tub 11, a sterilizing and cleaning tub 11 provided with a plurality of compartments 10r into which denture boxes 200 storing dentures are detachably put, and storing sterilizing and cleaning water; at least one bubble generator 12 for cleaning the dentures stored in the denture boxes submerged in the sterilizing and cleaning water by supplying air bubbles to each compartment of the sterilizing and cleaning tub.

The bubble generator 12 may apply any structure or theory to supply air bubbles to the sterilizing and cleaning water stored in the sterilizing and cleaning tub.

As an embodiment of the bubble generator 12, an air bubble generator or an air pump etc. used for a fish tank etc., as illustrated in FIG. 2, may be installed at one side of a housing, and an air hose may connect to the bottom of a water storage tank.

FIGS. 2 and 3 illustrate an embodiment where a bubble generator 12 consisting of an air bubble generator is installed at the lower portion of the device, but the bubble generator may be installed at the back or the upper portion of the device.

A bubble generator 12 of another embodiment may use an ultrasonic wave generator, and as illustrated in FIG. 3, the ultrasonic wave generator is installed at the bottom of the sterilizing and cleaning tub 11 and generates ultrasonic waves such that the sterilizing and cleaning water stored in the sterilizing and cleaning tub divides into minute bubbles and flows between artificial teeth thereby separating foreign substances from the teeth.

Such an ultrasonic wave generator may use one selected from the ones used for a dish washer or an air cleaner etc., but needs to have enough output to separate foreign substances left on dentures.

Preferably, air bubbles generated by the bubble generator 12 are separately supplied to each compartment 11r of the sterilizing and cleaning tub 11.

A bubble sterilizing cleaner 10 according to the present invention has a plurality of compartments, and the number of the compartments is not limited. Depending on the needs, some of the compartments may be used. When the dentures stored in some of the compartments are cleaned, supplying air bubbles to all the compartments causes waste of energy. Accordingly, the compartments are grouped into a predetermined number of compartments to be supplied with bubbles, and the wanted compartments operate thereby reducing waste of energy.

The bubble sterilizing cleaner 10 configured as in FIG. 2 and as described above, has compartments 11r in the sterilizing and cleaning tub 11 itself, and in FIG. 3, the sterilizing and cleaning tub 11′ itself consists of a space for storing sterilizing and cleaning water and has a box supporting means 13 with a plurality of compartments 13r to have the effect of installing a plurality of compartments.

The box supporting means 13, in which denture boxes 200 storing dentures are detachably installed, has a plurality of compartments 13r into which the denture boxes may be put.

As illustrated in FIG. 8, the denture box 200, on the outer circumferential surface thereof, has a stumbling projection such that the denture box is easily separated from the compartment when the denture box 200 is inserted into the compartment 13r formed in the box supporting means 13.

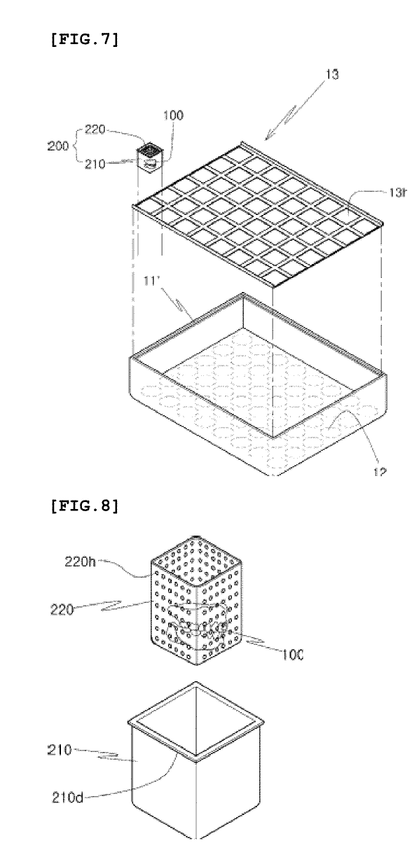

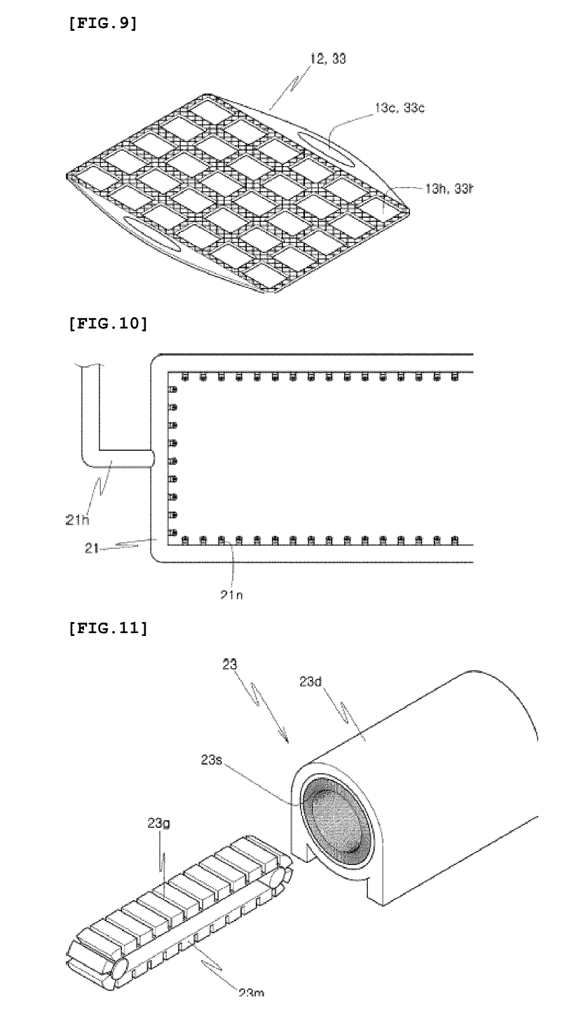

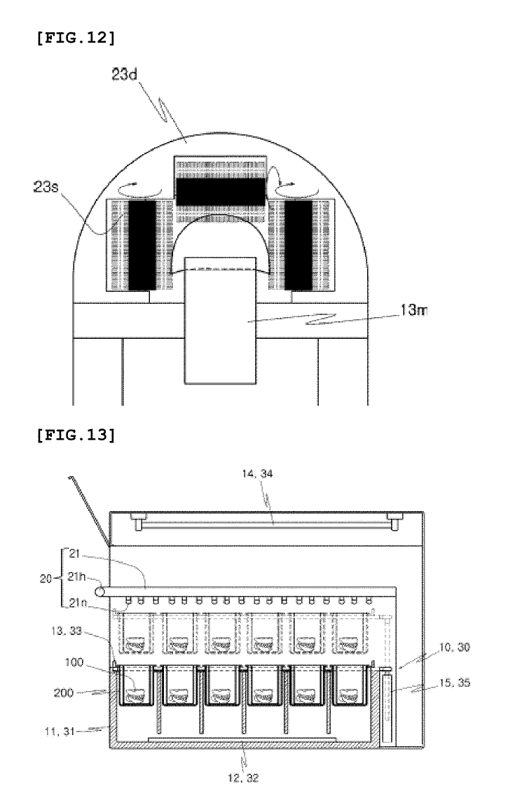

Such a denture box 200 may be configured as one, but preferably, is configured as two, as illustrated in FIGS. 7 and 8. That is, the denture box consists of an outer box 210 which has a stumbling projection 210d formed on the outer circumferential surface of the outer box, and an inner box 220 which is detachably installed in the outer box and has a plurality of holes 220h formed on the side wall of the inner box.

The net-type inner box is installed in the outer box, and the denture is stored in the inner box such that the denture may be taken out of the denture box without touching the sterilizing and cleaning water or the cleaning solution stored in the outer box by the hand.

The sterilizing and cleaning tubs 11, 11′ included in the bubble sterilizing cleaner 10 stores sterilizing and cleaning water as described above, and the sterilizing and cleaning water, in which water is mixed with germicide, or water is converted to have sterilizing ability, cleans the denture and at the same time sterilizes germs left on the denture.

That is, as illustrated in FIGS. 2 and 3, an antiseptic solution generator 17 is installed on a supply line for supplying water to allow the supplied water to have sterilizing ability.

The antiseptic solution generator 17 may consist of one or more of plasma generators, negative ion generators, ozone water generators or electrolysis devices.

The bubble sterilizing cleaner 10 may further include a light sterilizer 14, which is at the upper portion of the sterilizing and cleaning tubs 11, 11′ to sterilize germs left on the denture by irradiating light.

The light sterilizer 14 may use any one of the common ultraviolet lamps, infrared lamps or halogen lamps for killing microorganisms by using light such as infrared rays or ultraviolet rays etc. In particular, the ultraviolet ray lamp is allowed to sterilize germs left at the gap of the teeth of the denture by generating ultraviolet rays with 250 to 260 nm wavelengths having a strong sterilizing ability.

Further, the cleaning device, at one side thereof, may further have a display 15 to show advertisement descriptions or inform how to use the cleaning device.

The display 15, as illustrated in FIGS. 1 to 3, may be installed at the upper portion of the device or at one side of the device, and displays a manual of how to use a sterilizing and cleaning device for dentures according to the present invention and advertisements of companies manufacturing dentures.

Further, a sterilizing and cleaning device for dentures according to the present invention, as illustrated in FIG. 4, may further include a high pressure steam cleaner 20 and a sterilizing/storing means 30 around the bubble sterilizing cleaner 10.

The bubble sterilizing cleaner 10, the high pressure steam cleaner 20 and the sterilizing/storing means 30 may be separately configured, or as illustrated in FIG. 4, may be integrally configured.

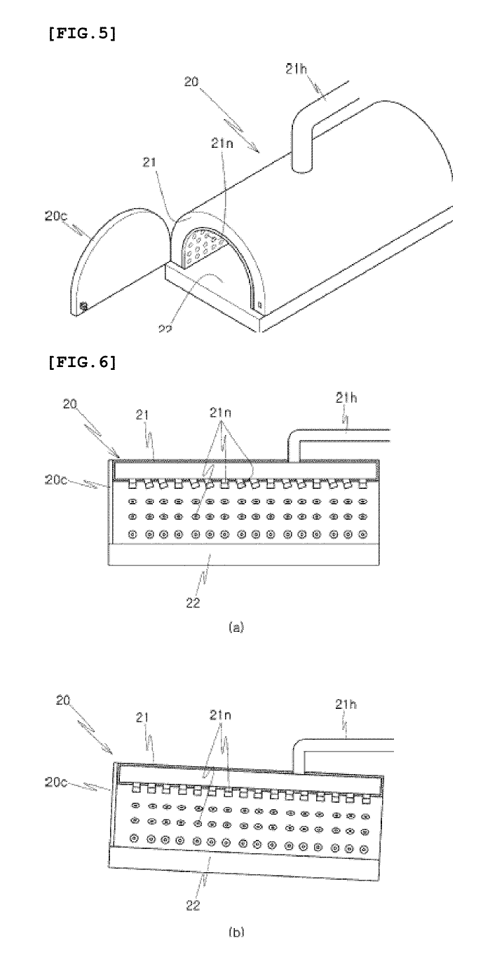

The high pressure steam cleaner 20, as illustrated in FIGS. 5 and 6, includes a steam spraying means 21, on the inner wall thereof, provided with a plurality of spraying nozzles 21n for spraying high pressure steam supplied through a supply hose 21h; and a supporter 22 installed at the lower portion of the steam spraying means 21 for supporting the denture.

As described above, the high pressure steam cleaner 20 may be integrally configured with other configuring devices as illustrated in FIG. 4, or may be separately configured as illustrated in FIGS. 5 and 6. In the case of the separate configuration, the steam spraying means 21, as illustrated in FIGS. 5 and 6, is configured to have a semicircle tubular body, to have double walls and to have a plurality of spraying nozzles 21n installed on the inner wall thereof such that high temperature and high pressure steam, supplied through the supply hose 21h, sprays through the spraying nozzles 21 inwardly to clean the denture placed on the supporter 22.

Such a high pressure steam cleaner 20 has a door 20c installed respectively at an opened inlet and outlet to prevent the steam sprayed inwardly from leaking outwardly, and the supporter 22, at one side of the bottom thereof, has a drainage (invisible) to discharge water in which steam is coagulated.

Further, the high pressure steam cleaner 20 is preferably configured to be long so as to simultaneously clean the dentures in large numbers, and in the case where the high pressure steam cleaner is made to be long, a separate tool for taking out and putting the dentures such as a denture tray is preferably installed on the supporter 22 to easily take out the cleaned dentures, or while being cleaned, the dentures 100 put in the high pressure steam cleaner preferably move toward the outlet such that a worker may take out the dentures.

One of the methods of helping a worker to easily take out the dentures cleaned in the high pressure steam cleaner 20 by moving the dentures toward the outlet is adjusting the direction in which the spraying nozzles 21n included in the high pressure steam cleaner 20 spray steam as illustrated in FIG. 6A. That is, a plurality of spraying nozzles are installed, but no less than half of the spraying nozzles spray steam toward the outlet such that the steam sprayed by the spraying nozzles cleans the dentures and at the same time, moves the dentures toward the outlet.

Another method of moving the dentures to the outlet is installing the high pressure steam cleaner 20 itself to incline toward the outlet as illustrated in FIG. 6B. By doing so, the dentures move toward the outlet by means of self-weight, and the spraying nozzles spray high temperature and high pressure steam on the moved dentures to separate foreign substances left on the dentures.

The high pressure steam sprayed by the high pressure steam cleaner 20 may separate foreign substances left on the dentures, but foreign substances in the gap between the artificial teeth might be left. In this case, it is preferable to brush the teeth.

Accordingly, a cleaning brush 23 is preferably further installed at the inlet or the outlet of the high pressure steam cleaner 20 to separate foreign substances from the dentures by causing friction on the dentures.

The cleaning brush 23 may be modified and embodied in various different forms. Preferably, however, the cleaning brush is to clean and easily move the dentures and at the same time not to damage the dentures. To this end, in a preferred embodiment as illustrated in FIG. 11, a brush 23b is rotatably installed in a tubular body 23d having the same external shape as the high pressure steam cleaner 20, and is configured to have a moving means 23m installed between the brush for moving the dentures.

That is, as illustrated in FIG. 11, the moving means is formed to have a conveyor shape, and while the conveyor operates in the state where the denture is inserted in and fixed to a denture fixing groove 23g of the moving means 23m and at the same time, high pressure water is supplied, the brush 23s rotates to brush foreign substances off the denture.

Preferably, the denture cleaned by the high pressure steam cleaner 20 moves to the bubble sterilizing cleaner 10 to be cleaned once again.

Further, a sterilizing and cleaning device for dentures according to the present invention preferably further installs a sterilizing/storing device 30.

The sterilizing/storing device 30, as illustrated in FIG. 4, is configured to include a sterilizing tub 31 which stores an antiseptic solution therein; one or more of antiseptic solution generators 32 which are installed at the bottom of the sterilizing tub for converting pure water into an antiseptic solution; a box supporting means 33 which is accessibly installed at the sterilizing tub and at which the denture box 200 storing the denture is detachably installed; and a light sterilizer 34 which is installed on the box supporting means and sterilizes germs left on the denture by irradiating light on the denture.

The sterilizing tub 31 is to provide a space for storing an antiseptic solution used for sterilizing, and in the case where the sterilizing tub, as illustrated in FIG. 6, is integrally configured, serves as a sterilizing and cleaning tub of the bubble sterilizing cleaner 10.

The antiseptic solution generator 32, which is used as a means to convert pure water stored in the sterilizing tub 31 into an antiseptic solution having sterilizing ability, may be modified and embodied in various different forms, but may consist of one or more of plasma generators, negative ion generators, ozone water generators or electrolysis devices which are commonly used for sterilizing.

In the case where the antiseptic solution generator 32 is a plasma generator, OH radicals having oxidizing and sterilizing abilities are generated by low temperature plasma to clean and sterilize the denture, and the sterilized and cleaned denture may be stored in the state where the denture is sterilized.

In the case where the antiseptic solution generator 32 is a negative ion generator, negative ions are generated in pure water to perform sterilization, and in the case where the antiseptic solution generator 32 is an electrolysis device, supplied electricity degrades foreign substances left on the denture and at the same time sterilizes the denture.

That is, the antiseptic solution generator 32 may be installed anywhere, as long as pure water stored in various sterilizing tubs may be converted into an antiseptic solution, and is used as a means to store the denture in a sterilized state, and sterilization is performed by the light sterilizer 34.

The light sterilizer 34 may use any one of the common ultraviolet lamps, infrared lamps or halogen lamps for killing microorganisms by using light such as infrared rays or ultraviolet rays etc. In particular, the ultraviolet ray lamp is allowed to sterilize germs left in the gap of the teeth of the denture by generating ultraviolet rays with 250 to 260 nm wavelengths having a strong sterilizing ability.

The box supporting means 33 may also use a box supporting means 23 included in the bubble sterilizing cleaner 20. That is, the box supporting means 23 moves to be equipped at the sterilizing/storing device 30 such that the denture doesn't need to be moved to another box supporting means.

A sterilizing and cleaning device for a plurality of dentures according to the present invention, as illustrated in FIG. 4, may be integrally arranged in a row, and in this case, the bubble sterilizing cleaner 10, the high pressure steam cleaner 20 and the sterilizing/storing means 30 are respectively configured to have the same structure described above.

Another embodiment of an integrated sterilizing and cleaning device for dentures may be configured as illustrated in FIG. 13.

In another embodiment of an integrated sterilizing and cleaning device for dentures as illustrated in FIG. 13, the high pressure steam cleaner 20 is movably installed at the upper portion of the sterilizing/storing device 30, the bubble sterilizing cleaner 10 is installed at the upper portion of the high pressure steam cleaner and uses the sterilizing tub, included in the sterilizing/storing device 30, as a sterilizing and cleaning tub 11 by storing sterilizing and cleaning water in the sterilizing tub, and the steam spraying nozzles 21n included in the high pressure steam cleaner 20 are installed at the upper portion of the box supporting means.

Because the bubble sterilizing cleaner 10 and the sterilizing/storing means 30 have a similar configuration, things that have the same functions may be configured as one as illustrated.

In the case of the integrated cleaning device, when being integrated as described above, a part of the cleaning device may have a different configuration. In terms of the difference in such a configuration, the high pressure steam cleaner 20, as illustrated in FIG. 10, is configured to install a plurality of spraying nozzles 21n at a water supply hose 21 consisting of a “⊏”-shaped pipe.

The spraying nozzles 21n, as illustrated, spray water inwardly in the direction in which the spraying nozzles face each other but downward in an inclined direction such that the sprayed water faces toward the denture box installed in the box supporting means.

Further, the water supply hose 21 is configured to have a “⊏” shape such that the box supporting means may ascend freely.

The above-described sterilizing cleaner 11 or sterilizing tub 31 are divided into a plurality of compartments to allow each denture box 200 installed in the box supporting means 13, 33 to come in and out of each compartment respectively such that cross contamination between the dentures may be reduced.

Even in the case where the bubble sterilizing cleaner 10, the high pressure steam cleaner 20 and the sterilizing/storing means 30 are integrally configured as described above, the first cleaning, the second cleaning and the sterilizing/storing processes need to be carried out in order, and in such processes, the location of the box supporting means 13, 11 needs to change.

To this end, the box supporting means 13, 33 is installed to be ascended by an ascending means 15, 35. When light is used for sterilization, the box supporting means ascends such that germs on the denture is sterilized by light such as ultraviolet rays or infrared rays etc. irradiated by the light sterilizer, and when the denture is stored, the box supporting means descends to be submerged in the antiseptic solution stored in the sterilizing tub and then, the denture is stored in the state where the denture is sterilized by ozone water, plasma ions, negative ions or electricity generated by the antiseptic solution generator 32.

That is, preferably, the ascending means 35 such as a hydraulic cylinder etc. is installed at the back of the device as illustrated in FIG. 4, a rectangular gap-shaped ascending platform ascended by the ascending means is installed, and the box supporting means is detachable at the ascending platform.

1. A sterilizing and cleaning device for a plurality of dentures for simultaneously sterilizing and cleaning a plurality of dentures (100) comprising:
a sterilizing and cleaning tub (11) provided with a plurality of compartments (10r) into which a denture box 200 storing a denture is detachably put, and storing sterilizing and cleaning water having sterilizing ability; and
a bubble sterilizing cleaner (10) comprising at least one bubble generator (12) for sterilizing and cleaning the denture stored in the denture box submerged in the sterilizing and cleaning water by supplying air bubbles to each compartment of the sterilizing and cleaning tub.

2. A sterilizing and cleaning device for a plurality of dentures for simultaneously sterilizing and cleaning a plurality of dentures (100) comprising:
a sterilizing and cleaning tub (11′) storing sterilizing and cleaning water therein;
at least one bubble generator (12) cleaning a denture stored in a denture box submerged in the sterilizing and cleaning water by supplying air bubbles to each compartment of a box supporting means, described hereunder, installed at the sterilizing and cleaning tub; and
a box supporting means (13) having a plurality of compartments (13r) which are accessibly installed at the sterilizing and cleaning tub and at which the denture box (200) storing the denture is detachably installed.

3. The sterilizing and cleaning device for a plurality of dentures according to claim 1 or 2,
wherein the sterilizing and cleaning tubs 11, 11′, at an upper portion thereof, further comprises a light sterilizer 14 sterilizing germs left on the denture by irradiating light on the denture.

4. The sterilizing and cleaning device for a plurality of dentures according to claim 3,
wherein the cleaning device, at one side thereof, further comprises a display (15) showing advertisement descriptions or informing how to use the cleaning device.

5. The sterilizing and cleaning device for a plurality of dentures according to claim 4,
wherein the bubble sterilizing cleaner (10), at one side thereof, further comprises a high pressure steam cleaner (20) separating foreign substances left on the denture by spraying high pressure steam on the denture (100), and
wherein the high pressure steam cleaner (20) comprises a steam spraying means (21) provided with a plurality of spraying nozzles (21n) on an inner wall of the steam spraying means to spray high pressure steam supplied through a supplying hose (21h); and a supporter (22) installed at a lower portion of the steam spraying means (21) to support the denture.

6. The sterilizing and cleaning device for a plurality of dentures according to claim 5,
the sterilizing and cleaning device for a plurality of dentures further comprising a sterilizing/storing device (30),
wherein the sterilizing/storing device (30) comprises a sterilizing tub (31) storing an antiseptic solution therein; one or more of antiseptic solution generators (32) installed at one side of the sterilizing tub; a box supporting means (33) which is accessibly installed at the sterilizing tub and at which the denture box (200) storing the denture is detachably installed; and a light sterilizer (34) sterilizing germs left on the denture by irradiating light on the denture.

7. The sterilizing and cleaning device for a plurality of dentures according to claim 6,
wherein the high pressure steam cleaner (20) is movably installed at an upper portion of the sterilizing/storing device (30), the bubble sterilizing cleaner 10 is installed on an upper portion of the high pressure steam cleaner and uses the sterilizing tub, which the sterilizing/storing device (30) comprises, as a sterilizing and cleaning tub (11) by storing sterilizing and cleaning water in the sterilizing tub, and
wherein the high pressure steam cleaner (20) comprises steam spraying nozzles (21n) installed at an upper portion of the box supporting means such that the steam spraying nozzles 21n are installed between the bubble sterilizing cleaner (10) and the antiseptic solution generator (32), and the box supporting means (33) is installed to ascend by using an ascending means (35) such that the denture may move among the sterilizing and cleaning tub, the light sterilizer and the steam spraying nozzles to be cleaned and sterilized.

8. The sterilizing and cleaning device for a plurality of dentures according to claim 6,
wherein when light is used for sterilization, the box supporting means (33) is installed to be ascended by an ascending means (35), such that germs on the denture are sterilized by light irradiated by the light sterilizer, and when the denture is stored, the box supporting means descends to be submerged in the antiseptic solution stored in the sterilizing tub, and afterwards, the denture is stored in the state where the denture is sterilized by the antiseptic solution converted by the antiseptic solution generator (32).

9. The sterilizing and cleaning device for a plurality of dentures according to claim 6,
wherein the sterilizing tub (31) is divided into a plurality of compartments, and the box supporting means (33) is configured to have a net body formed with denture box installing holes (33h) such that the denture box is put into each compartment.

10. The sterilizing and cleaning device for a plurality of dentures according to claim 5,
wherein the supporter (22) is formed to incline from an inlet toward an outlet or installs a conveyor, such that the denture moves toward the outlet while foreign substances are cleaned by means of steam, or
wherein no less than half of the spraying nozzles sprays steam toward the outlet, such that the denture moves towards the outlet while foreign substances are cleaned by means of steam.

11. The sterilizing and cleaning device for a plurality of dentures according to claim 5,
wherein the high pressure steam cleaner (20), at an inlet or outlet side thereof, further comprises a cleaning brush (23) for separating foreign substances from the denture by causing friction against the denture, and
wherein the cleaning brush (23) has a brush 23b rotatably installed in a tubular body (23d), and a moving means (23m) for moving the denture there between.
